(19) United States
(12) Patent Application Publication
(10) Pub. No.: US 2007/0048795 A1
(43) Pub. Date: Mar. 1, 2007

(54) Immunoaffinity separation and analysis compositions and methods

(76) Inventors: Xiangming Fang, San Diego, CA (US); Lei Huang, San Diego, CA (US); Wei-Wei Zhang, San Diego, CA (US)

Correspondence Address: Xiangming Fang, 6777 Nancy Ridge Drive, San Diego, CA, 92121, US

(21) Appl. No.: 11/508,517

(22) Filed: Aug. 23, 2006

Related U.S. Application Data
(60) Provisional application No. 60/712,002, filed on Aug. 26, 2005.

(51) Int. Cl. G01N 33/53; C12M 1/34

(52) U.S. Cl. 435/7.1; 435/287.2

(57) ABSTRACT

The present invention provides compositions and methods for immunoaffinity separation of targets from mixtures for enrichment, identification, quantification, and analysis. In particular, disclosed are avian IgY antibodies coupled to solid supports and their methods of use. Further disclosed are systems and methods for fractionating or enrichment a mixture of biological materials in an automated multiplex and high-throughput platform or system.

The present application claims priority to U.S. Provisional Patent Application No. US60/712,002, filed Aug. 26, 2005, the disclosure of which is incorporated by reference herein in its entirety.

### FIELD OF THE INVENTION

The present invention relates to immunoaffinity separation and analysis of biological materials. The invention further relates to compositions of affinity reagents linked to solid supports and the methods that the solid supports mediate affinity reagents to separate or enrich targets from non-targets in mixtures of biological samples. More specifically, the present invention relates to polyclonal avian yolk immunoglobulin (IgY) antibody compositions and methods of making and using them. The compositions and methods according to the present invention are useful for immunoaffinity capture, separation, enrichment, identification, analysis and characterization of a protein target or a complex mixture of biological materials. Additionally, the compositions and methods according to the present invention can be subject to multiplexed or high-throughput processes for a plurality of mixtures of biological materials.

### BACKGROUND OF THE INVENTION

IgY antibody is immunoglobulin isolated from egg yolks of the lower vertebrates, such as birds, reptiles, and amphibians (Leslie, G. A. & Clem L. W., J Exp Med 1969, 130, 1337-1352; Hadge, D. & Ambrosius H., Mol Immun 1984, 21, 699-707; Du Pasquier, L., et al., Annu Rev Immunol 1989, 7, 251-275). Avian IgY antibodies have been developed and successfully applied for various types of immunoassays (Larsson, A. & Mellerstedt H., Hybridoma 1992, 11, 33-39; Larsson, A., et al., Poultry Science 1993, 72, 1807-1812; Warr, G. W., et al., Immunol Today 1995, 16, 392-398; Schade, R. & Hlinak A., ALTEX 1996, 13, 5-9; Zhang, W.-W., Drug Discovery Today 2003, 8, 364-371). An outstanding advantage of avian IgY antibodies is that they are secreted by hens into egg yolk, resulting in a high-yielding reservoir of easy-to-access antibodies (Patterson, R., et al., J Immunol 1962, 89, 272-278). Compared to drawing blood, collecting eggs is non-invasive, continuous, convenient, and scalable. One egg yolk contains about 100 mg of total IgY. After a primary injection and three boosts, one hen can produce 40-60 eggs, yielding about 5 grams of antibodies. Distinct from IgG antibodies in molecular structure and biochemical features, IgY antibodies were shown to have several advantages over IgG, particularly for their high avidity and less cross-reactivity to human proteins (Stuart, C. A., et al., Anal Biochem 1988, 173, 142-150; Gassmann, M., et al., FASEB J 1990, 4, 2528-2532; Larsson, A., et al., Clin Chem 1991, 37, 411-414). Unlike IgG, the IgY Fc region does not bind human proteins such as complements, rheumatoid factor, Fc receptor, IgM, etc, significantly increasing IgY's specificity of capture.

Affinity-purified polyclonal IgY antibodies were conjugated in an oriented fashion to a solid phase such as to develop highly specific immunoaffinity capture reagents for protein separation. These IgY microbeads (known as Seppro™) were developed in both individual and mixed formats. Compared to IgG-based microbeads, IgY microbeads were shown to have higher avidity and more specific binding of target proteins (Fang, X., et al, Drug Discovery Today: Technologies 2004, 1, 141-148; Huang, L., et. al., Proteomics 2005, 5, 3314-3328).

The proteome of a given tissue or cellular system has enormous biochemical diversity and a wide dynamic range of protein concentrations to carry out various biological and physiological functions of the system. This wide range of protein concentrations can be several orders of magnitude beyond the detection range of the currently most useful protein separation and identification methods, such as two-dimensional gel electrophoresis (2DE), liquid chromatography (LC) and mass spectrometer (MS). “Divide and conquer” is a strategy that has been articulated to cope with the overwhelming dynamic range of protein concentrations present in proteomes or complex protein mixtures. To appropriately manage the analysis of protein diversity and dynamic range, a proteome must be partitioned into sub-proteomes, and a complex protein mixture must be further fractionated, to purify (or partition) and measure target proteins. Affinity separation is one of the most specific and effective approaches for partitioning or fractionating protein mixtures or complex biological solutions.

To enhance detection and quantification of proteome components, it is advantageous to prepare biological samples via specific pre-fractionation or depletion methods, which separate highly abundant components from those of medium or low abundance. Immunoaffinity separation or fractionation of proteins has proven to be one of the most effective approaches for this purpose, and many products are currently commercially available. However, the currently unmet demand in biomedical research is how to divide, partition or fractionate a complex protein sample in a global or systematic way. “Seppromics” is a new term to describe this concept and approach, which represents a rapidly-developing area in fractionation, separation or partitioning of proteins or biological materials.

In seppromics, separation, depletion, partitioning, or fractionation are designed and organized for quality, representative, reproducible, and consistent sample preparation for the purpose of specific, accurate and reliable analysis of proteins and related materials. In general, single-step partitioning of a highly complex protein mixture is usually far from sufficient to dissect and analyze the sample. For example, due to the wide dynamic range of protein concentration in human plasma, specific removal of the top 6 to 12 highly-abundant proteins (HAP) from human plasma by immunoaffinity partitioning results in isolation of the next level of abundant protein: moderately abundant proteins, MAP. The MAP then becomes a new challenge for effectively and accurately detecting and analyzing low-abundant proteins (LAP) (Omenn, G S. et al., Proteomics 2005, 5, 3226-3245).

Besides separation and reduction of complexity of biological materials, especially for those with a huge dynamic range of protein concentration, how to effectively and specifically capture and enrich target proteins is another challenge in proteomic discovery and analysis. Enrichment is needed for isolating specific protein targets from a mixture, which can be one of the applications of immunoaffinity separation. IgY-mediated immunoaffinity can be well used for capturing or enriching protein complex and can be applicable for “interactome” (Vidal, M., Cell 2001, 104, 333-339), which describes the protein-protein interactions and their relationship with other components in a proteomic context.

To make protein fractionation or enrichment robust, reproducible, and more controllable, automation and multiplex of the immunoaffinity separation processes will be very useful in high-throughput handling of large volume and quantity samples, which can enable comprehensive studies of samples from a group of subjects or a population. IgY microbeads were previously made in liquid chromatographical column forms and can be used in a semi-automated process (Huang, L., et. al., Proteomics 2005, 5, 3314-3328), but the throughput is relatively low. More robust formats such as multi-well and microtip with liquid handler can be one of the solutions to increase the throughput of automation. In addition, how to effectively analyze (i.e. identify and quantify) the fractionated components after multiplex high-throughput immunoaffinity separation treatment is also an unmet need in proteomic profiling, discovery, and validation studies. It is also the challenge that this present invention addresses.

### BRIEF SUMMARY OF THE INVENTION

Briefly, in a specific embodiment of the present invention, an affinity separation composition is provided which is comprised of affinity reagents linked to a solid support and the methods that the solid support mediates affinity reagents to separate targets from non-targets in mixtures of biological samples. The term “separation” or “separate” used herein refers to partitioning, depletion, fractionation, isolation, removal, reduction of a part in a mixture, and enrichment of a part in a mixture. The separation process can be a serial fractionation process or an enrichment process. The process can be automated, in a multiplex form for high-throughput process, and followed by analytical methods such as mass spectrometry to identity or measure targets. A preferred embodiment of the affinity reagents employed in the present invention is a polyclonal antibody composition of Immunoglobulin Yolk (IgY antibody) having an Fc region and Fab antigen binding regions. The IgY antibody composition comprises a solid support covalently linked to oxidized glycosylation moieties in the Fc region of the polyclonal IgY antibodies wherein the Fab regions of the IgY polyclonal antibodies are capable of reacting with an antigen. The present invention also includes the above described polyclonal IgY antibody composition that additionally contains an antigen bound or hybridized to the Fab antigen binding regions of the antibody. The IgY antibodies can be individually immobilized to a solid support in a single or mixture format, or immobilized to a solid support as a mixture in certain ratio of the antibodies. The immobilized IgY antibodies, being placed in various types of holders or devices and applicable for the processes described above, are useful for immunoaffinity capture, separation, enrichment, purification, and detection of a desired protein target in a complex mixture.

One aspect of the present invention was developed in response to meet the challenge of global and systematic protein fractionation and represents a novel and useful approach to biological sample preparation and proteomic partitioning. The methods described herein represent ways to fractionate moderately-abundant protein (MAP) from highly-abundant protein (HAP), or LAP from MAP or HAP and therefore enrich or concentrate the relative amount of low-abundant protein (LAP) in plasma, where the majority of biologically interesting and commercially important biomarkers reside. These methods have important applications in complex protein mixtures, samples subjected to proteomics analysis, plasma/serum, other body fluids, subcellular fractions, tissue and cell culture extracts, and other sub-proteomes. In particular, the embodiments of the present invention describe immunoaffinity protein fractionation or enrichment that can be used in a single-step or in multiple sequential steps. The methods described herein are readily adaptable to different formats and scales of protein separation by using suitable devices or carriers and different combinations of fractionation or enrichment methods. The unique biochemical and immunological features of these methods enable further development for immunoaffinity capture, fractionation, enrichment, detection, and analysis. The methods described in the present invention can also be combined with other protein fractionation products to better meet the needs of scientists and provide solutions to facilitate protein biomarker discovery and validation.

One of the examples is to partition the MAP fraction, which remains after removal of the HAP, from LAP. The immunoaffinity column used (SuperMix column) for this purpose was developed using IgY microbeads directed to capture the immunogenic proteins in a flow-through fraction of a MIXED12 column (Huang, L., et. al., Proteomics 2005, 5, 3314-3328), where the top 12 most highly abundant human plasma proteins were specifically depleted. The SuperMix column is then used as a means of further partitioning the MIXED12 flow-through to remove or enrich the MAP, which will substantially enrich the LAP in the flow-through fraction of the SuperMix column. The merit of this approach is that it is global and systematic, and does not require purified or individual protein antigens. This approach utilizes the animal's native immune system to develop large groups of antibodies directed against this complex antigen mixture of moderately abundant proteins, and to subsequently use the immobilized mixture of purified antibodies (SuperMix column) to specifically capture and partition the proteins or the targets of the antibodies in biological samples. This process can then be repeated, using proteins in the flow-through fraction of the first SuperMix column to immunize new animals, and to immobilize the resulting purified polyclonal antibody preparation to produce a “SuperMix-2” column. The SuperMix-2 flow-though fraction can be further developed in a repeated process to partition lower and lower abundance proteins. The SuperMix columns can also be combined in a flexible way for the purpose of fractionation and enrichment of a given complex mixture.

The polyclonal IgY compositions of the present invention can be used in multiplex format (microtip or multi-well) with high-throughput sample processing equipment, such as Magtration System (PSS Bio Instruments). This format can be widely used by industrial-scale proteomics companies or in clinical laboratories for multiplex and high-throughput sample preparation for downstream proteomic analysis by identification or quantification methods such as mass spectrometer.

Another aspect of the present invention includes a kit or a functional unit wherein the kit or the functional unit comprises an immunoaffinity separation composition (e.g., the polyclonal IgY composition) or a plurality of immunoaffinity separation compositions.

### BRIEF DESCRIPTION OF THE FIGURES

FIG. 1—Basic composition and process of affinity separation. Listed are the various elements, components and materials that can be used to enable the composition and process of affinity separation of specific targets from a mixture containing non-specific targets.

FIG. 2—Variations of basic compositions of affinity separation. Diagrams depict two examples of variations of the basic composition and process shown in FIG. 1. A, is shown to use the molecular affinity bridge, e.g. biotin and avidin or streptavidin, to link an affinity reagent to solid support. B, multiple affinity reagents (e.g. IgY antibodies) mixed in a certain ratio first, then linked to solid support, different from that in FIG. 1, where one affinity reagent is linked to solid support.

FIG. 3—Basic Composition and Method of SuperMix. Human plasma is first separated from 12 HAP via MIXED12 column, which contains IgY microbeads against the 12 HAP. The flow-through fraction of the MIXED12 column is used as a mixture of antigens for immunizing chickens and generating polyclonal IgY antibodies. This fraction is called the 12 HAP-Depleted Fraction or F1 fraction, comprising moderate and low abundant proteins, MAP/LAP. The 12 HAP-Depleted Fraction is also used as an affinity ligand and conjugated to a solid support (e.g., agarose) for packing into an antigen affinity column (Antigen-Affinity Column), which is used to purify the SuperMix antibodies from the total IgY isolated from the chickens immunized with the 12 HAP-Depleted Fraction. The SuperMix antibodies are conjugated to microbeads and packaged into the SuperMix Column. The 12 HAP-Depleted Fraction is then applied to the SuperMix column. Two fractions, flow-through (F2) and eluted (E), are obtained and further characterized by proteomics techniques well known in the art.

FIG. 4—Protein Partitioning by SuperMix Column. Human plasma samples were applied to a Seppro™ MIXED12-LC20 column. The flow-through fraction (F1) was collected and directly loaded onto a SuperMix LC2 column. The flow-through fraction (F2) was collected and bound proteins were eluted. The materials bound to the SuperMix column were eluted (E). The samples were subject to SDS PAGE analysis. It is important to note: (1) there is little or no overlap between the F2 and E proteins, indicating the effective partitioning of the SuperMix column; and (2) F1 and E protein patterns are very similar, indicating effective immunization of chickens with the HAP-Depleted Fraction and successful production of the SuperMix column.

FIG. 5—Two Dimensional Electrophoresis (2DE) Analysis of Protein Fractions of SuperMix Column. Human plasma samples were applied to a Seppro™ MIXED12-LC20 column. The flow-through fraction (F1) was collected and directly loaded onto a SuperMix LC2 column. The flow-through fraction (F2) was collected and bound proteins were eluted. The materials bound to SuperMix column were eluted (E). Then 100 μg samples were subject to 2DE analysis. The results clearly indicated that the detected proteins from fraction E were different from these detected from fraction F2, which further confirmed that the SuperMix column indeed efficiently partitioned the fraction F1 from IgY12.

FIG. 6—Resolution of Fractions of SuperMix Column for Mass Spectrometry Analysis. SuperMix flow-through and elution fractions were used. Each fraction was loaded into six wells (10 μg of proteins per well) and a total of 60 μg of proteins were therefore analyzed for each SuperMix fraction. After electrophoresis, each gel lane was divided into 30 sections for the eluate fraction and 31 sections for the flow-through fraction. Each section was subjected to mass spectrometry analysis.

FIG. 7—Example of IgY-Microbead Tip (SepproTip) Design. Shown is SepproTip-IgY12 design and specification. This design is based upon the microtip type developed by PSS Bio Instruments. The example tip size is 500 μl packed bed volume.

FIG. 8—Example of SepproTip-IgY12 and Automated Liquid Handler. Two pictures are shown. A. A 500 μl SepproTip-IgY12 packed in the tip of PSS Bio Instruments. B. Two SepproTips mounted on the Magtration System 6GC (PSS Bio Instruments) during sample processing.

FIG. 9—SepproTip-IgY12 Protein Fractionation Process. The diagram shows the process flow that has been successfully used for SepproTip-IgY12 application in fractionating human plasma.

FIG. 10—Efficiency of SepproTip-HSA in Removing Target. To analyze the capacity and fractionation efficiency of SepproTip-IgY, SepproTip-HSA was used to remove HSA in human plasma. A1: Two volumes (40 μl and 48 μl) of samples were loaded to SepproTip-HSA. ELISA assay was used for analyzing the flow-through fractions to determine the capture and removal efficiency. A2: 40 μl sample loaded, HSA depleted at 99.7%; A3: 48 μl sample loaded, HSA depleted at 99.6%; B2: 40 μl sample loaded, HSA depleted at 99.7%; B3: 48 μl sample loaded, HSA depleted at 99.3%.

FIG. 11—Example of SepproTip-IgY12 Fractionating Plasma Sample. To further analyze the specificity, capacity, efficiency and reproducibility of immunoaffinity fractionation, SepproTip-IgY12 (500 μl) was used to process 20 μl of human plasma. Shown are the flow-through fractions from 10 consecutive runs and one representative elution fraction.

FIG. 12—Comparison of Separation Efficiency of Two Types of SepproTip-IgY. To test efficiency of IgY immunoaffinity separation based upon different types of immobilized IgY, IgY antibodies were covalently linked to a solid support via two types of preparation. One type is SepproTip-IgY12, prepared by linking 12 individual IgY antibodies covalently to beads first, then mixing them in a certain ratio. Another type is SepproTip-IgYm12, prepared by mixing 12 individual IgY antibodies in a certain ratio first, then covalently linking them to beads. Both tips (500 μl bed size) were tested under the same conditions to fractionate 20 μl human plasma. The results demonstrate that SepproTip-IgYm12 has better capture efficiency and capacity.

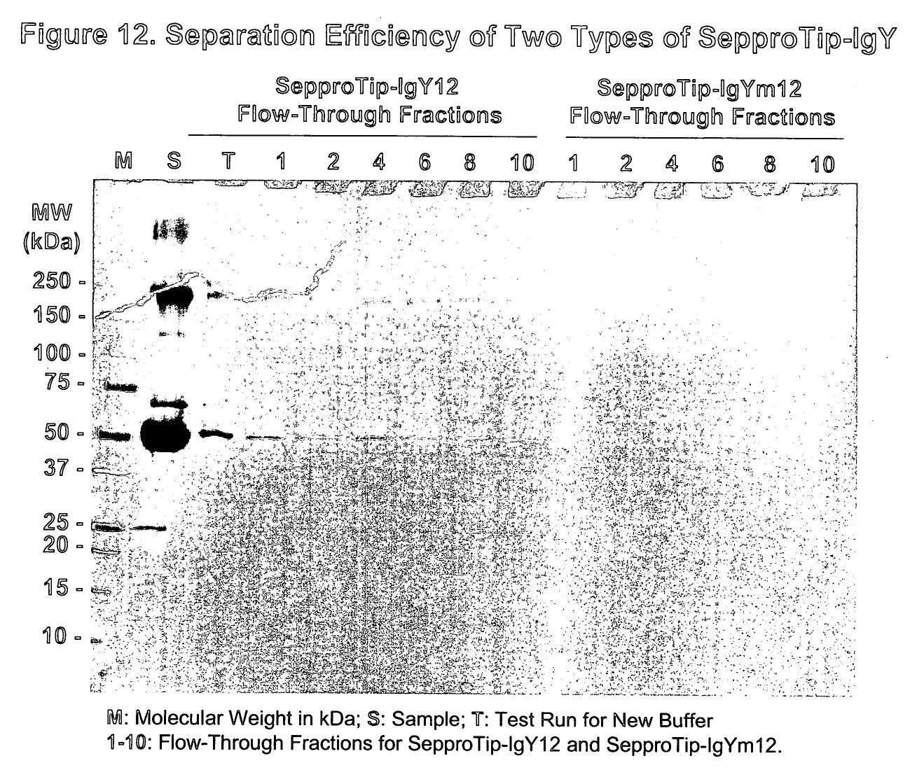

FIG. 13—Sequential Immunoaffinity Partitioning (SIP) Using Reiterated SuperMix Method. Shown is a process of successive fractionation of protein mixtures to access lower and lower abundance proteins. A complex mixture is first used as antigens (or immunogens) to immunize chickens (Immunization Host-1) to develop a SuperMix-1 column (as shown in FIG. 18). The initial complex mixture is applied to the SuperMix-1 column and results in Elution-1 and Flow-Through-1. The latter Flow-Through-1 fraction is then used as antigens to immunize additional chickens (Immunization Host-2) to generate SuperMix-2 column. The Flow-Through-1 is then applied to a SuperMix-2 column and results in Elution-2 and Flow-Through-2. The process can be repeated until the original complex mixture is fully partitioned. As shown by using Elution-2 fraction to generate antibodies, the eluted fractions can also be used as antigens for immunization to generate a plurality of antibodies, wherein these antibodies specifically bind to the eluted fractions and can be used as SuperMix for fractionating antigens in the eluted fractions from a proteome or sub-proteome or enriching certain antigens or biological materials therefrom. The types of samples that can be used for SIP processes and the host system that can used for developing affinity reagents are listed. The SIP processes can also be combined with other fractionation and analytical methods such as 2DE, LC/MS/MS, PF 2D, etc.

### DETAILED DESCRIPTION OF THE INVENTION

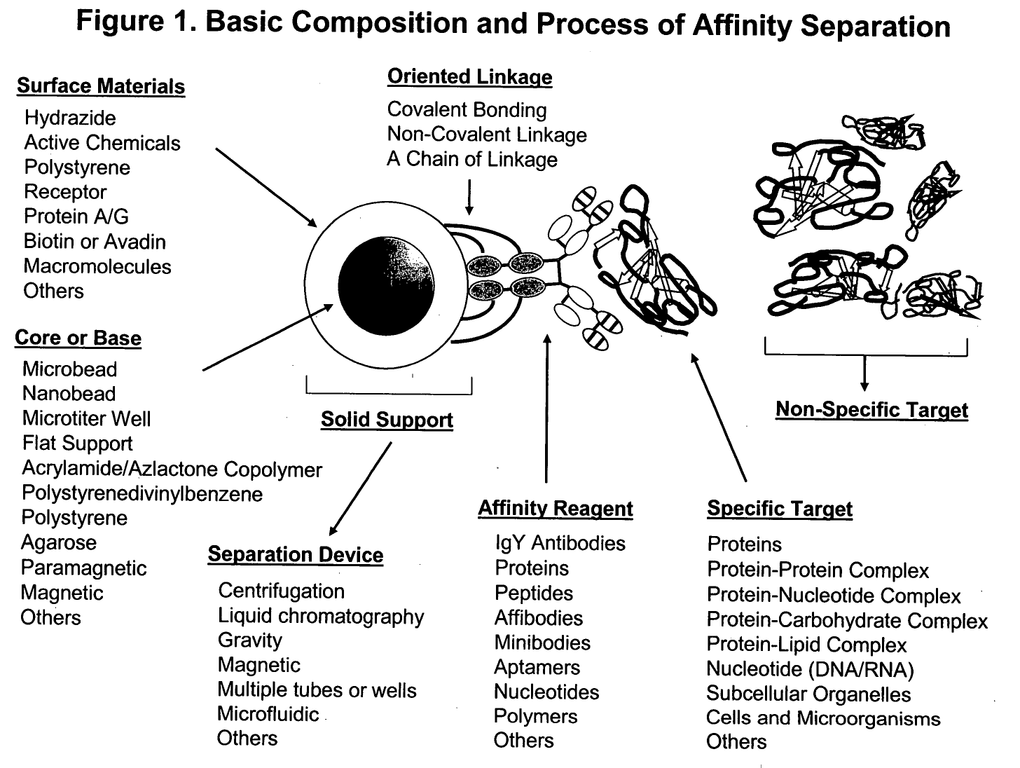

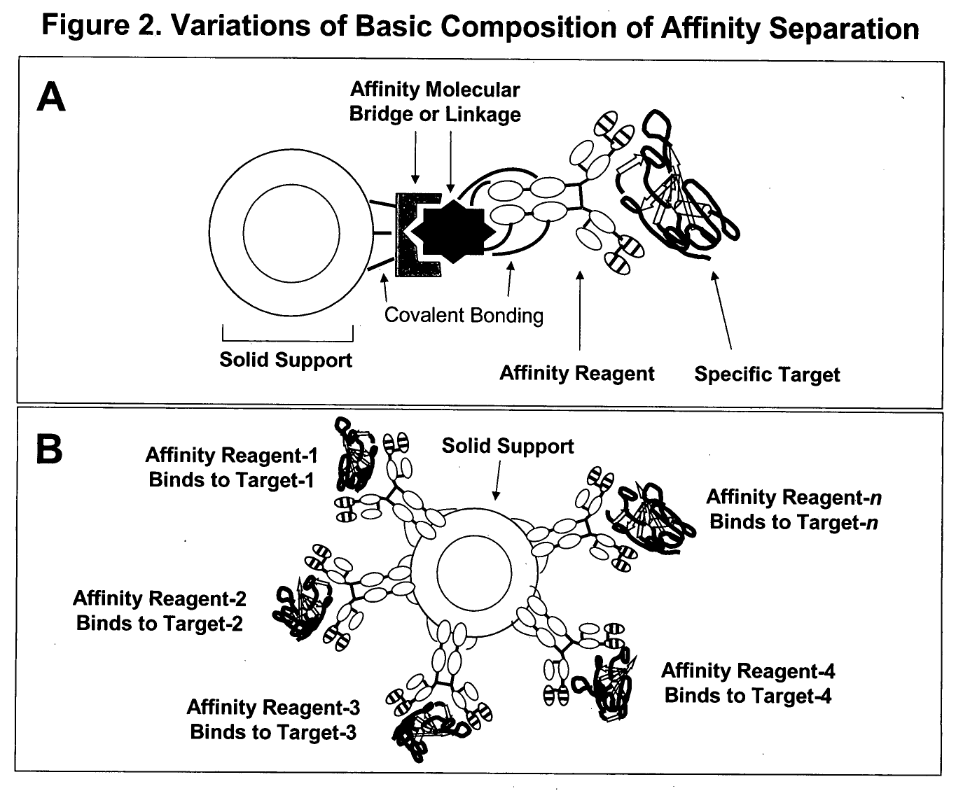

In practicing the present invention, the following basic components, processes, and variations (FIGS. 1 and 2) are employed to conduct an affinity separation process:

Affinity Reagents—These are biological substances or macromolecules that can specifically bind to targets through affinity recognition and attractive forces between reagents and targets. Affinity recognition, resembling the relationship between lock and key, is highly specific for the target and usually has a dissociation constant below 10⁻⁸ M (Winzor D. J., J Chromatogr. 2004 1037(1-2): 351-67; Chaiken I. M., J Chromatogr. 1986, 376: 11-32). The affinity reagents can include IgY antibodies, proteins, peptides, affibodies, minibodies, aptamers, nucleotides, polymers and others.

Separation—This is a process or a series of processes to separate, fractionate or divide a complex mixture. The separation process can be used for sample preparation or enrichment of certain targets, and also is a means of partition, depletion, fractionation, isolation, purification, and/or removal or enrichment of certain target(s) from the total mixture.

Fraction—means one or more parts of the total components of a complex mixture which are a result of an initial complex mixture after being processed (e.g., through a separation process) and quantitatively different or separated from the initial complex mixture.

Enrichment—means one or more parts of the total components of a complex mixture that are concentrated, enhanced, increased, isolated or purified from the complex mixture.

Flow-Through Fraction—means the fraction of a complex mixture that does not bind to an immunoaffinity composition under certain conditions.

Elution Fraction—means the fraction of a complex mixture that contains proteins or elements that bind to an immunoaffinity composition under certain conditions and then are eluted or separated from the immunoaffinity composition by applying elution conditions.

IgY polyclonal antibody—means gamma globulins derived from the egg yolk of an avian species.

Avian species—refers to any bird, preferably chickens (Gallus gallus).

Antigen—means any compound that is recognized and specifically bound by the polyclonal antibody preparation. Typically, this same antigen is used to immunize the bird for producing polyclonal antibodies in the yolk. The immunization is typically done by injecting a bird with a purified antigen. In the case of protein antigens, a bird can be injected with polynucleotides that can express the protein antigen or immunogenic portions thereof thereby making the antigen in situ in the bird.

Specific Targets—These are also biological materials, macromolecules, molecules, or complexes. The specific targets are usually antigens that can induce antibodies in animals. The specific targets can also be other materials such as proteins, protein-protein complexes, protein-nucleotide complexes, protein-carbohydrate complexes, protein-lipid complexes, nucleotides (DNA/RNA), subcellular organelles, cells and microorganisms and others. The specific targets are usually mixed or complexed with other non-specific targets. The specific targets that bind specifically to affinity reagents can be separated from those non-specific targets in a given mixture of specific targets and non-specific targets.

Covalently linked—when referring to IgY antibodies, means oriented conjugation of the IgY antibodies with the antigen binding fragment available for antigen binding. This occurs by oxidizing the IgY-Fc glycosylation moieties, converting hydroxyl groups to reactive aldehyde groups, which then react with chemical groups on the solid support forming stable covalent bonds.

Oriented Linkage—These are the chemical or biological materials that can link affinity reagents to the surface of solid supports. Linkages can be covalent bonds between the affinity reagents and the surface of the solid support. Linkages also can be indirect, through a chain of covalent bonding and non-covalent affinity binding, as shown in FIG. 2.

Solid Support—These are the materials that are attached to the affinity reagents through oriented linkage and can mediate the affinity reagents to separate bound targets from those non-specific targets. The solid support generally comprises surface materials and a core or base. The surface materials are the active chemical or biological materials that can link the solid support to the affinity reagents. These materials comprise hydrazide, active chemicals, polystyrene, receptor, protein A/G, biotin, avidin, strepavidin, macromolecules and others. The core or base is coated with the surface materials and linked to affinity reagents via surface materials. The core or base can be the materials that help or mediate the separation of that affinity reagent-target complex. Examples of the core or base include microbeads, nanobeads, microtiter wells, flat supports, acrylamide/azlactone copolymer, polystyrenedivinylbenzene, polystyrene, agarose, paramagnetic, magnetic and others.

Immobilized IgY—means IgY antibodies that are linked or fixed to a solid support.

Separation Devices—These are the forces, attractions, apparatus, or processes that mediate the separation of the affinity reagent-target bound solid support from mixture of targets or biological materials. Examples of separation devices include gravity, centrifugation, liquid chromatography, magnetic force, multiple tubes, tips, wells, microfluidic, and others.

The basic composition and process of affinity separation specified in the present invention are depicted in FIG. 1 with some examples of related materials. The composition and process can be engineered into different variations. FIG. 2 depicts two classes of variations:

Variation 1—Shown in FIG. 2A, the linkage of affinity reagents to solid support is indirect, which is designed to have a molecular bridge, a pair of affinity reagents such as biotin and avidin. Each end of the molecular bridge is fixed to the affinity reagent or solid support through covalently bonding. This is a type of chain linkage, where the link can be combinations of covalent or non-covalent associations.

Variation 2—The solid support can be attached to affinity reagents in a different way. Shown in FIG. 2B, the solid support is bound to a group of affinity reagents mixed at a given ratio before the linkage process takes place. The ratio of mixing of affinity reagents is based upon the optimized binding of the affinity reagents to targets and on the effectiveness of affinity separation.

Affinity Separation Composition or Immunoaffinity Separation or Enrichment Composition—One or more affinity reagents linked to a solid support and oriented in a manner to facilitate the activity of the affinity reagents, whereas the affinity reagents are capable of binding specific targets by affinity recognition and the solid support is capable of mediating separation of the affinity reagent-target complex from a mixture. In one embodiment, the composition contains IgY antibodies linked or fixed to a solid support. In another embodiment, the composition contains IgY antibodies covalently linked to a solid support through the Fc region of the IgY antibodies.

Kit or Functional Unit—Immunoaffinity separation compositions can be placed, organized or assembled into a kit or a functional unit. The kit or the functional unit may further include a substrate or in a holder, or a plurality of substrates or holders. The holder can be a column, a pipette tip, a microtip, a tube, or a fluidic channel. In one embodiment, the kit or the functional unit may contain an immunoaffinity separation composition on a substrate or in a holder. In another embodiment, the kit or the functional unit may contain a plurality of immunoaffinity separation compositions whereas the compositions are in different regions of a substrate or in different holders which are arranged in array, multiplex, or parallel. Further, the kit or the functional unit may contain a plurality or substrates or holders whereas each substrate or holder having at least one immunoaffinity separation composition.

Highly-Abundant Proteins (HAP)—The set of proteins found in the highest concentration in a given biological sample that typically interferes with the sensitive detection of less abundant proteins. In serum or plasma, HAP includes those proteins with initial concentrations between about 50 mg/ml and about 1 ug/ml, and are comprised of a set of at least 12 proteins collectively accounting for up to 96% of the total protein mass, including Serum Albumin, IgG, Fibrinogen, Transferrin, IgA, IgM, Haptoglobin, α2-Macroglobulin, α1-Acid Glycoprotein, α1-Antitrypsin and HDL (Apo A-I & Apo A-II). These proteins are specifically captured by the Seppro™ MIXED12 immunoaffinity partitioning columns.

Moderately-Abundant Proteins (MAP)—The set of proteins found in a middle range of concentrations in a given biological sample, which remain along with the Low Abundant Proteins after specific removal of the HAP. In serum or plasma, MAP includes those proteins with the initial concentrations between about 5 ug/ml and about 10 ng/ml. This set of proteins is primarily used for immunization of chickens and preparation of antigen affinity columns to partition the MAP from the LAP. Some biomarkers fall in this set of proteins.

Low-Abundant Proteins (LAP)—The set of proteins found in a low range of concentrations in a given biological sample, which is in the flow-through fraction from a SuperMix column. In serum or plasma, LAP typically includes those proteins with the initial concentrations below about 10 ng/ml, and is often the source of commercially important biomarkers often accessible only by direct immunological assay, such as ELISA or Western Blot. The technology described in this application permits characterization of a large set of LAP using physical methods, such as mass spectrometry.

SuperMix—The mixture or group of antibodies that are developed against a mixture of proteins and related materials as antigens in a given immune host or system. The mixture of antibodies can be generated depending upon the relative concentrations and strength of immunogenicity of the antigens in a complex mixture. The SuperMix contents can be specific or can vary from time to time, in different conditions or preparations.

SepproTip—IgY microbeads are packed in pipette tips or in the microtips used in automated liquid handlers. The tips are designed to have a bottom and atop frit, and filters or membrane screens to hold packed IgY microbeads in between. The IgY microbeads packed in tips can be individual or mixed antibodies. For example, SepproTip-IgY12 is the tip that is packed with MIXED12-IgY microbeads; and SepproTip-Mix is the tip that is packed with IgY-SuperMix microbeads. SepproTip can be used in a manual manner or in an automated setting on a liquid handler, which can be multiplexed for high-throughput processing of large numbers or volumes of samples.

Seppromics—This is a concept or approach of protein separation from a proteomics perspective. In accordance with the concept of “interactome” (Vidal, M., Cell 2001, 104, 333-339), which describes the protein-protein interactions and their relationship with other components in a proteomic context, seppromics considers observing and preserving the natural, interactive, or functional relationships of the members in the complex protein mixture during the separation or partitioning process of the protein mixture. In this consideration, separation, depletion, partitioning, or fractionation of the protein solution or complex mixture are designed and organized in a global and systematic manner. The process will take into account of the natural or structural (hierarchical) relationship of the components in the protein mixture or proteome to best retain the original biological or physiological conditions for high quality, representative, reproducible, and consistent sample preparation. This is to best meet the purpose of specific, accurate and reliable analysis of the proteins and related materials isolated from a complex mixture. The global and systematic approach comprises sequential, repetitive, or combinatorial partitioning of a protein solution or complex mixture by developing proper immunoaffinity materials.

Sequential Immunoaffinity Partitioning (SIP)—This is one of applications of the seppromics approach and a reiterated process of the SuperMix method. To systematically partition a complex mixture or to divide a proteome into a group of sub-proteome fractions, tandem SuperMix antibody groups can be generated by applying a column of the first group of antibodies (SuperMix-1) to obtain the flow-through fraction (or flow-through eluent) that can be used as a mixture of antigens for generating the next group of antibodies (SuperMix-2), which can be linked to solid phase (microbeads) and packaged into the second column. The process can be repeated as many times as desired until a proteome is fully partitioned. The eluted fractions (or eluent which contains a mixture of antigens binding to the SuperMix) from each SuperMix columns can be used as enriched materials for proteomics analysis. The eluted fractions can also be used as antigens for immunization to generate a plurality of antibodies, wherein these antibodies specifically bind to the eluted fractions and can be used as SuperMix for partitioning antigens in the eluted fractions from a proteome or sub-proteome or for enriching certain antigens or biological materials therefrom.

In one embodiment of the present invention, an affinity separation composition for separating one or more target compounds present in a complex mixture is made by linking an affinity reagent to a solid support oriented in a manner to facilitate the activity of the affinity reagents or its ability to further react with a target. Once prepared, the affinity separation composition is contacted with a complex mixture to remove the target from the mixture by affinity recognition of the target by the affinity reagent. The solid support component of the affinity separation composition mediates the separation of the affinity reagent-target complex from the complex mixture. The resulting complex mixture has a reduced level of the target and preferably no detectable levels of the target. The affinity reagent-target complex can then be processed to strip the target so that the affinity separation composition can be re-used. Additionally, the target can be recovered and/or analyzed to determine if there is an association between other materials and the target.

More specifically, in practicing the present invention, polyclonal IgY antibodies can be covalently conjugated to a solid support material by oxidizing the glycosylation moieties in the Fc region of the polyclonal IgY antibodies and then reacting oxidized antibodies with a solid support material that has reactive moieties that will form a covalent bond (conjugation) with the oxidized glycosylation moieties. This reaction forms an antibody composition that orients the antigen binding region away from the support material and allows the antibody to react with an antigen.

The solid support can be in any physical configuration such as beads or membranes. However, any configuration that increases the surface area of the solid support is preferred because an increased surface area will allow for more attachment sites of the IgY antibody in a given volume. For this reason, beads, including nanobeads, are a preferred solid support configuration. Beads can be in a pre-packed or batch mixture format. Beads can also be used in a continuous process format. Magnetic and paramagnetic beads can also be employed as the solid support to aid in the separation or enrichment of the polyclonal IgY beads after being contacted with the complex protein mixture.

If a solid support material is used that will react specifically with the Fab regions of the IgY antibody, then the support material can be coated to render the material non-reactive to the Fab regions and facilitate a reaction with the Fc region of the IgY antibody. For example, polystyrene beads, including styrene nanobeads, can be coated with avidin or streptavidin to prevent reactions between the polystyrene and the Fab regions on the antibody. The avidin coated polystyrene beads are then reacted with biotin that has been modified to contain hydrazide groups that can then react with the Fc region of the IgY antibody. This allows for the proper orientation of the IgY antibody for maximum efficiency in hybridizing with the desired protein (antigen) in the complex protein mixture. In another example, periodate-oxidized IgY is reacted with a bifunctional linker molecule containing a hydrazide at one end and a ligand at the other end. The resulting IgY-ligand molecule then binds tightly and specifically with a ligand receptor bound to a solid surface, such as a microbead. The linker molecule is bifunctional and comprises a hydrazide moiety at one end to bind to the Fc region of the IgY and biotin at the other end, which serves as the ligand to bind to the solid support. Coupling of the biotinylated IgY to a solid surface is mediated through avidin or streptavidin which coats the underlying solid surface.

The immunoaffinity separation or enrichment composition can be held or carried in different types of separation devices, which can be in various forms or formats on different types of platforms or in different types of systems. The spin column (SC) and liquid chromatographic column (LC) are widely used formats. The pipette tip, microtip, multi-well plate, bead or chip array, microfluidic chip, etc. are also devices that are suitable as the application form of the immunoaffinity separation or enrichment composition. It is important and desirable to make sure that the separation devices are compatible for the platform or system that allows automation, multiplex and high-throughput processes. One of the embodiments of the present invention describes the SepproTip form and another embodiment describes the SuperMix Columns format. Both are examples of the immunoaffinity separation or enrichment composition that are applicable for the purpose of automation and/or multiplex for controllable, reproducible, and high-throughput performance needs.

Once the polyclonal IgY antibody composition (collectively referred to hereinafter as “present IgY composition”) is prepared, it can be used to capture desired protein target(s) from a complex protein mixture. This is done by contacting or incubating a sample of the complex protein mixture with the present IgY composition. The depleted sample can be recovered and contacted with a fresh or recycled batch of the present IgY composition one or more additional times depending on the binding capacity and protein concentration of the sample. The sample is then analyzed by identification or quantification methods such as mass spectrometer to determine if all of the desired protein has been removed from the sample. Additionally, after the separation is complete, the present IgY composition used in the separation or enrichment reaction can be treated to strip the desired protein target(s) from the antibodies, which can then be analyzed to determine if other proteins or materials are associated with the desired protein target(s).

The exact amount of polyclonal IgY antibody composition used in practicing the present invention (immunoaffinity separation or enrichment process) is not critical as any available IgY antibody will react with the target protein. Excess amounts of IgY antibody are employed if all of the target protein is to be removed from the complex protein mixture. If less than all of the target protein is to be removed from the complex protein mixture then the amount of IgY antibody is adjusted accordingly. Routine titration experiments can be conducted to determine the optimum amount of antibody needed per weight of target protein.

In human serum separation with the present polyclonal IgY composition it is desirable to remove at least about 95% by weight and preferably at least about 98% of the high abundant proteins. For human serum albumin (HSA) removal at least about 99% and preferably at least about 99.9% or more is removed from the complex protein mixture.

When mixtures of different IgY antibodies are used to separate or enrich multiple target proteins from a complex mixtures, the ratio of the different IgY antibodies should preferably approximate the ratio of the target proteins present in the complex protein mixture. Routine analytical procedures (ELISA, Western blot, etc.) can be employed to determine the ratio of target proteins present in the complex protein mixture and then the corresponding IgY antibody ratios are calculated and mixed accordingly. For example, if HSA and IgG are the target proteins in a serum sample and upon analysis of the serum sample are present in a weight ratio of 4:1 (HSA/IgG), then it would be preferred to employ an IgY antibody composition that contains about 80% anti-HSA IgY antibodies and 20% anti-IgG IgY antibodies (4:1 ratio) in amounts effective to react with substantially all of the target proteins present in the complex mixture. If other target proteins are to be removed from the serum, then the ratios of all of the target proteins are calculated and the specific IgY antibodies are prepared in accordance with the calculated protein ratios.

In another embodiment of the present invention, an affinity separation composition for fractionation or enrichment, an undefined multiple or mixed target compounds present in a complex mixture is based upon a mixture or group of antibodies or affinity reagents linked to a solid support oriented in a manner to facilitate the activity of the antibodies or affinity reagents or their ability to further react with targets. This is an approach of seppromics, which applies sequential or combinatorial fractionation of a proteome or a complex mixture in a natural, global, and systematic manner. The seppromics approach can be applied to systematically fractionate a proteome through the SIP approach, which specifically removes a group of targets step-by-step by developing a group of antibodies against relatively abundant proteins in the sample of the proteome and applying the antibodies as immunoaffinity reagents to capture and remove the target proteins. The process can be repeated and multiple sub-proteomic fractions can be obtained.

More specifically, an example of using the SIP approach to systematically partition human plasma is to develop SuperMix columns and use them to sequentially remove specific groups of proteins from human plasma. The MIXED12 column contains IgY microbeads against 12 top abundant human plasma proteins: Albumin (HSA), IgG, Fibrinogen, Transferrin, IgA, α2-Macroglobulin, IgM, α1-Antitrypsin, Haptoglobin, α1-Acid Glycoprotein (Orosomucoid), Apolipoprotein A-I and Apolipoprotein A-II or High Density Lipoprotein. The MIXED12 column can specifically remove the 12 HAP by one-step immunoaffinity partitioning (Fang, X., et al, Drug Discovery Today: Technologies 2004, 1, 141-148; Huang, L., et. al., Proteomics 2005 5, 3314-3328). Thus, the moderately abundant proteins (MAP) and less abundant proteins (LAP) are substantially enriched after removal of the 12 HAP.

The first SuperMix column was developed using IgY microbeads against the immunogenic proteins in a flow-through fraction of MIXED 12 column, which was depleted of the top 12 HAP. The SuperMix column was then used as a means of further partitioning of the MIXED 12 flow-through to remove or enrich the MAP, which made the LAP more visible and measurable in the flow-through fraction of SuperMix column. An important advantage of this approach is that purified or individual protein antigens are not needed for specific immunoaffinity partitioning. This approach uses the chicken's immune system to develop groups of antibodies in a single step based upon the relative concentration and immunogenicity of the proteins or immunogens in the mixture, and to use the immobilized mixture antibodies (SuperMix column or other format) to specifically capture and partition the proteins or the targets of the antibodies. The SuperMix-2 fraction can be further developed using the SuperMix-1 column as a repeated process of the first fraction of the SuperMix. The SuperMix columns can also be combined in a flexible way for the purpose of fractionation and partitioning of a given complex mixture.

The eluted fractions of SuperMix column are an enriched group of proteins that may have specific features or a relationship of specific protein complex. The eluted proteins can also be further used as antigens to develop antibodies. This can generate a group of antibodies that are more selective and can be well defined when the protein identities of the eluted fraction are determined by some analytical methods such as mass spectrometry (MS). The antibodies generated from a specific elution fraction of the SuperMix column can be used as immunoaffinity reagents for capture, separation, fractionation, and detection of their targets.

The SIP process can continue to generate multiple fractions, which can be flow-through or elution from a SuperMix column at any given step. The fractions are subject to further processes and analyses, for example, 2DE and MS can be used for identifying proteins in the fractions. This is an approach that can systematically partition a proteomic sample and dig deeper into the proteome to overcome the hurdles of proteomic profiling and identification caused by the wide dynamic range of protein concentration.

The purpose of immunoaffinity separation or enrichment is to identify, detect, measure, analyze, profile, or quantify known or unknown protein target(s) in a complex biological mixture. These processes are significant for life science and biomedical applications. The IgY-based immunoaffinity separation or enrichment can enable these processes. In another embodiment of the present invention, the immunoaffinity separation or enrichment approaches can be combined with other methods or instruments of separation, fractionation, enrichment, isolation, detection or analysis. SuperMix, SIP, or SepproTip methods can be combined by LC and MS such as Strong Cationic Exchange (SCX), 2DE, or PF 2D (ProteomeLab PF 2D system, Beckman Coulter). More specifically, immunoaffinity separation or enrichment followed by SCX fractionation coupled directly to Reversed Phase Capillary (RPC) columns and LC-MS products (RPC-MS-MS) can simplify complex protein digest characterization for increased detection productivity. Whether Multidimensional Chromatographic Analysis of Proteins (MudPIT), Signature Peptides, the DALPC method (Direct Analysis of Large Protein Complexes), ICAT™ (isotope-coded affinity tagged) labeled, or specific amino acids containing peptides are desired from digested proteins for proteomic characterization, IgY-based immunoaffinity separation plus SCX column in tandem with a capillary RPC column allows true 2D chromatographic fractionation of more than 2000 peptides per run at more than 5 orders of magnitude better detectability than simple 2DE. In one embodiment of the present invention, by using SuperMix coupled with LC/MS/MS, it was shown, with the assistance of IgY immunoaffinity separation or enrichment, previously undetectable protein target(s) using 2DE or MS under similar conditions became detectable, demonstrating the utility and application potentials of IgY-immunoaffinity separation or enrichment for proteomic studies.

Following are examples that illustrate procedures for practicing the invention. These examples should not be construed as limiting its scope. All percentages are by weight and all solvent mixture proportions are by volume unless otherwise noted.

### EXAMPLES OF THE INVENTION

### Example 1

### SuperMix Column Development and Test

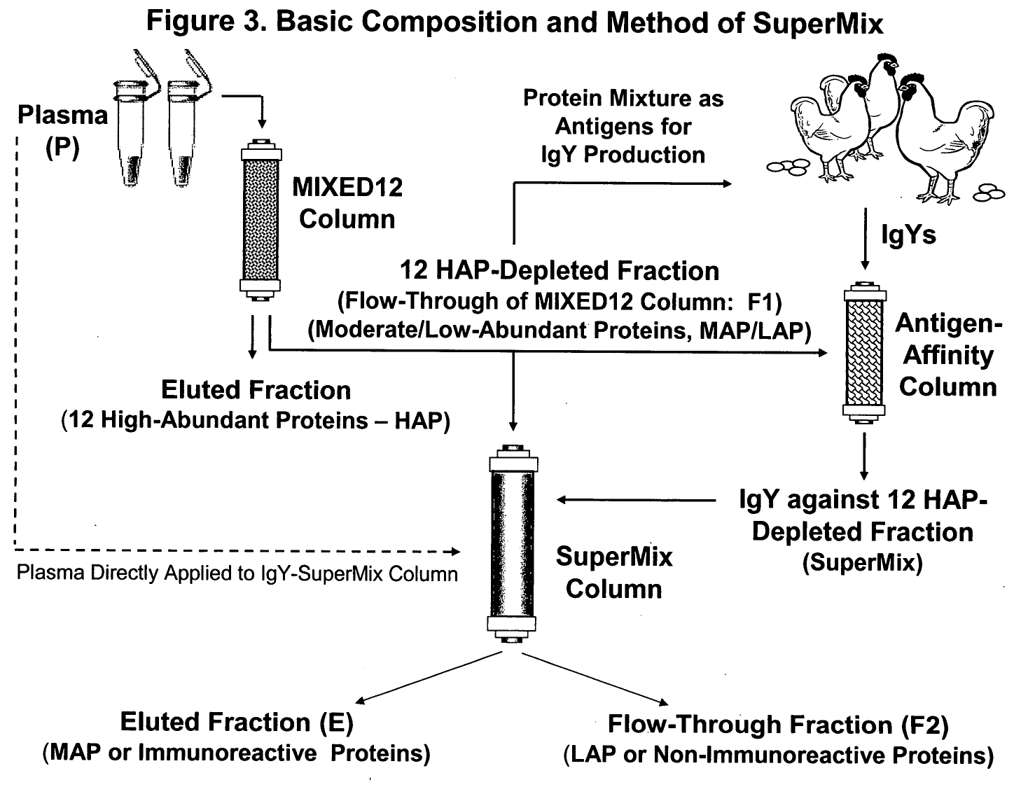

1. Generation of SuperMix and Affinity Column (FIG. 3)

The Seppro™ MIXED12 LC20 column was used to fractionate human plasma (Sigma). 200 μl of plasma sample was loaded to the column per run. The flow-through fraction with removal of 12 abundant proteins was collected. Proteins were concentrated using 3K MWCO spin column (Vivascience). The protein solution was used as immunogen to immunize chickens and as ligand for affinity-purification. IgY antibodies were isolated from eggs and affinity-purified through affinity chromatography. Purified IgY antibodies were named as SuperMix. SuperMix is conjugated to UltraLink Hydrazide Gel (Pierce Biotechnology, Inc., Rockford, Ill.).

2. Immunoaffinity Separation and Enrichment with Seppro™ MIXED12 and SuperMix

2.1 One-Dimensional Gel Electrophoresis (SDS-PAGE)

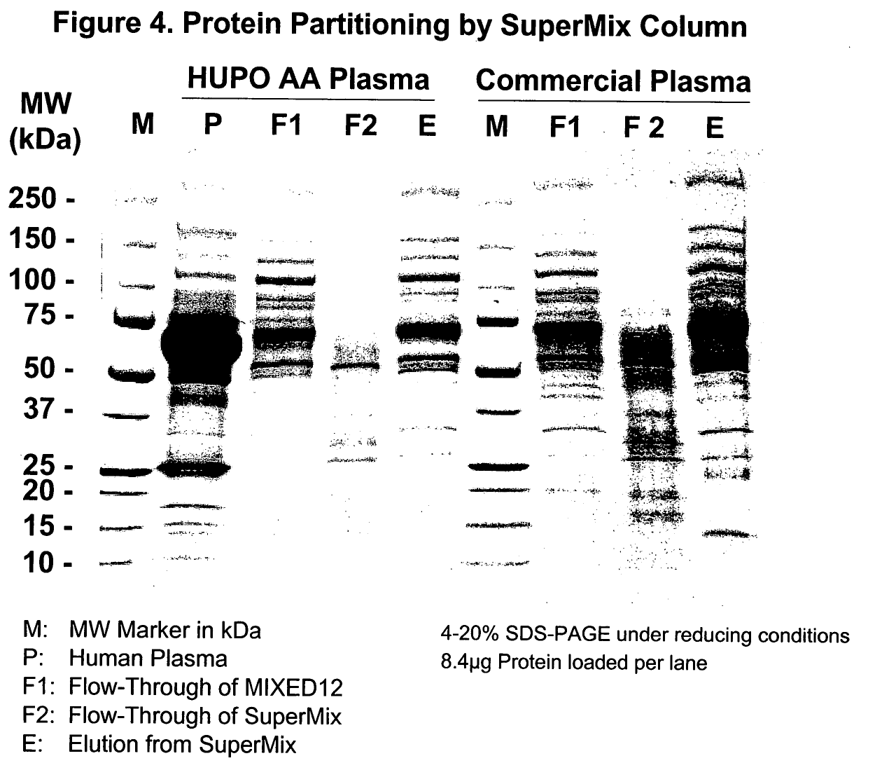

Human plasma samples were diluted in TBS and loaded to Seppro™ MIXED12 LC20 column. The flow-through fraction (F1) was collected and directly loaded onto IgY-SuperMix LC2 column. The flow-through fraction (F2) was collected and bound proteins were eluted with 0.1M Glycine, pH 2.5. Both F1 and F2 fractions were concentrated using 3K MWCO spin column (Vivascience). The materials bound to SuperMix LC2 column (2 ml packed bed volume) were eluted (E). The samples and fractions were subject to SDS PAGE analysis. It is important to note in FIG. 4: (1) there is little or no overlap between the F2 and E proteins, indicating the effective partitioning of the SuperMix column; and (2) F1 and E protein patterns are very similar, indicating effective immunization of chickens with the HAP-Depleted Fraction and successful production of the SuperMix column.

2.2 Two-Dimensional Gel Electrophoresis (2DE)

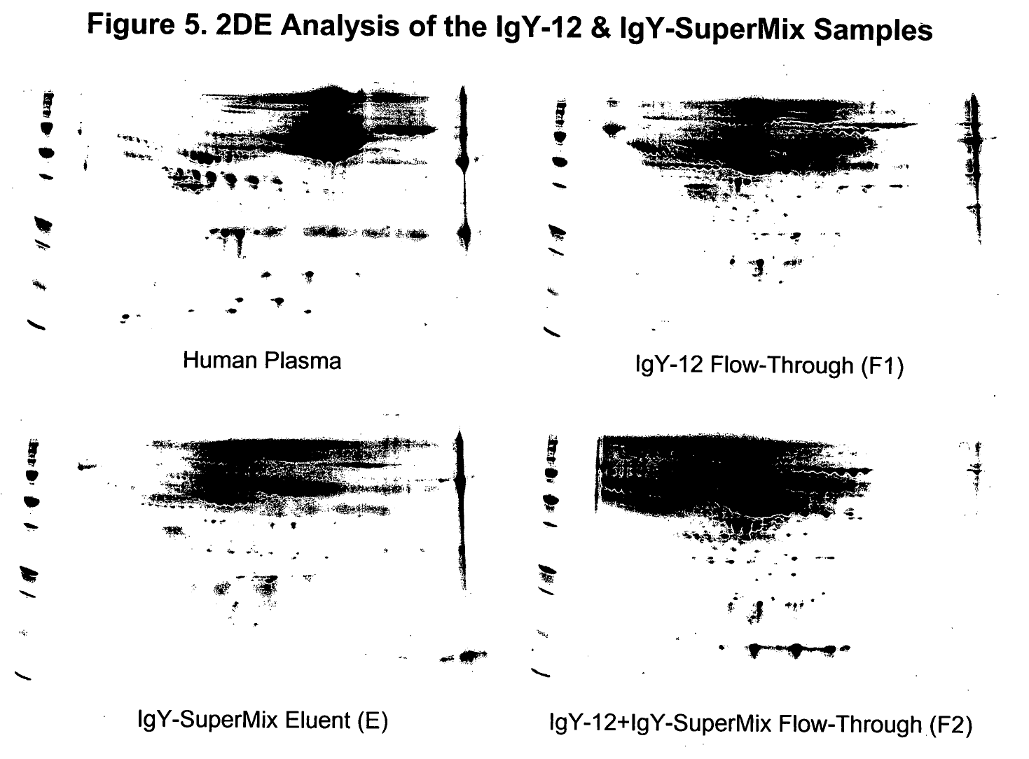

Fractions of Seppro™ MIXED12 LC20 and IgY-SuperMix LC columns (2 ml packed bed volume) are collected as described in 3.1. Prior to isoelectric focusing (IEF), samples were solubilized in 40 mM Tris, 7 M urea, 2 M thiourea and 2% CHAPS, reduced with tri-butylphosphine, and alkylated with 10 mM acrylamide for 90 minutes at room temperature. Following an acetone precipitation, the pellet was again solubilized and buffer exchanged in 7 M urea, 2 M thiourea, and 2% CHAPS until conductivity is <150 uS/cm, and 100 μg protein were subjected to IEF on 11 cm pH 3-10 and pH 4-7 immobilized pH gradient (IPG) strips (Amersham). Following IEF, IPG strips were equilibrated in 6 M urea, 2% SDS, 50 mM Tris-acetate buffer (pH 7.0), and 0.01% bromophenol blue and subjected to SDS-PAGE on 8-16% Tris-HCL Gel (Bio-Rad). All gels were stained in Sypro (r) Ruby (Molecular Probes, Eugene, Oreg.) and imaged by a charge-coupled device camera on a fluorescent imager (Bio-Rad Gel-Doc). Results are shown in FIG. 5.

2.3 Mass Spectrometry Analysis.

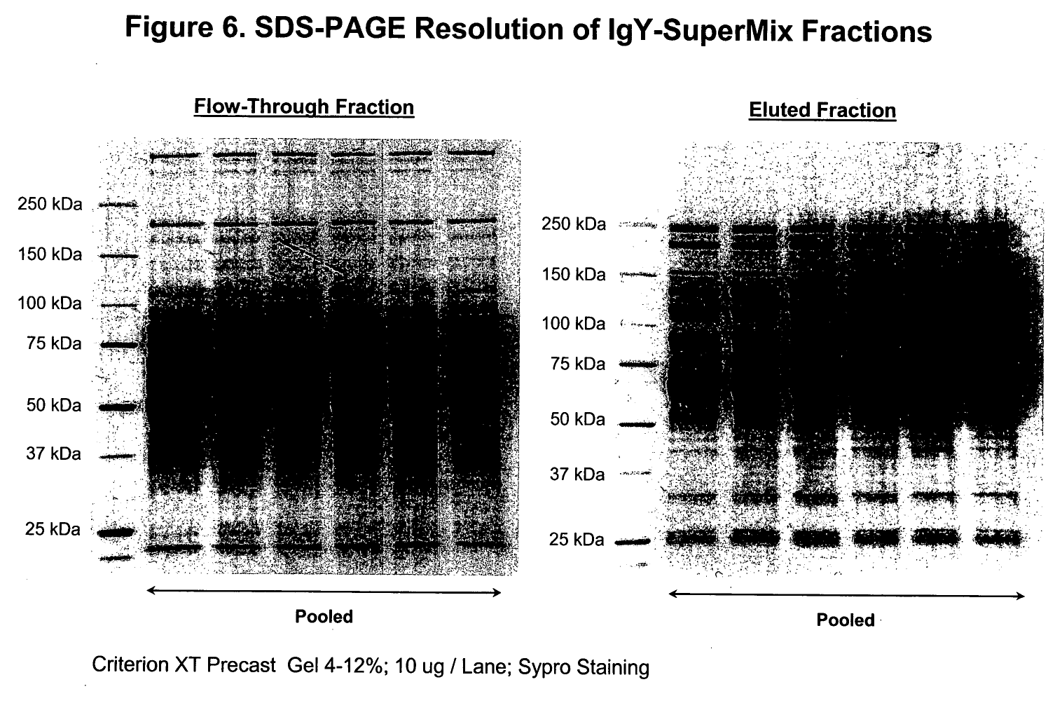

Again, fractions of Seppro™ MIXED12 LC20 and IgY-SuperMix LC2 columns are collected as described in 3.1. SuperMix flow-through and elution fractions were used for Mass Spectrometry Analysis. Each fraction was loaded into six wells (10 μg of proteins per well) and a total of 60 μg of proteins were therefore analyzed for each SuperMix fraction. After electrophoresis, each gel lane was divided into 30 sections for the eluate fraction and 31 sections for the flow-through fraction. The gel pictures are shown in FIG. 6 and proteins were stained with Sypro Ruby fluorescent protein stain. In-gel protein reduction, alkylation, and digestion were performed and peptide extracts were analyzed using a NanoLC 1100 series system (Agilent) coupled with a hybrid quadrupole-TOF mass spectrometer (QSTAR xl, MDS Sciex) equipped with a nano-electrospray ionization source (Proxeon) fitted with a 10-μm-ID fused-silica tip (FS360-20-10-D, New Objective). From initial data analysis, 81 proteins with high-confidence (two or more unique peptides) were identified in SuperMix eluted fraction. There are 130 proteins identified with low-confidence (one unique peptide) in this fraction. In the flow-through fraction from IgY-12/SuperMix columns, there are 126 proteins identified with high-confidence and 198 proteins with low-confidence. Among identified proteins, there are low abundance or cellular proteins, such as TGFβ-induced 68 kDa and Neural cell adhesion molecule 1 (flow-through), RNA-binding protein 6 and Eukaryotic translation initiation factor 3 (eluted).

### Example 2

### SepproTip Design and Testing

1. Example Design of SepproTip and Automated (Multiplex and High-Throughput) Process.

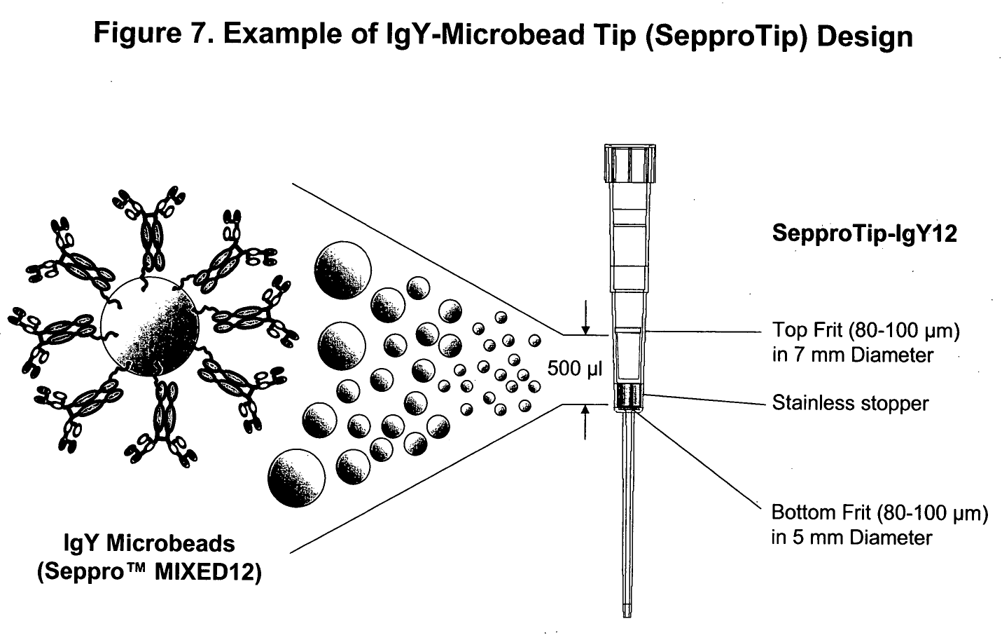

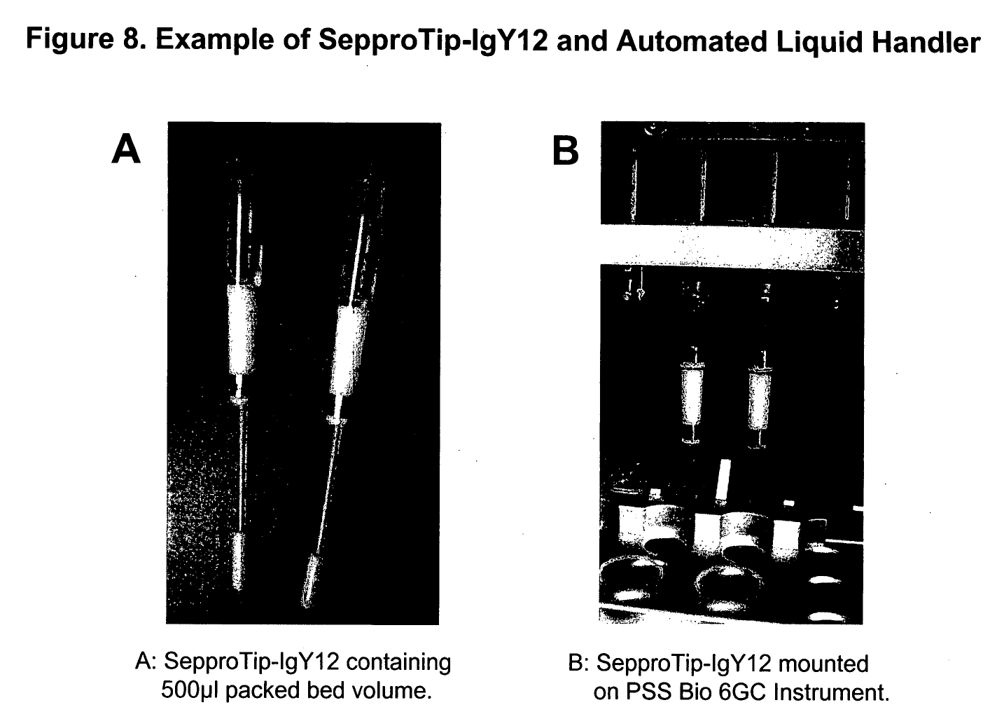

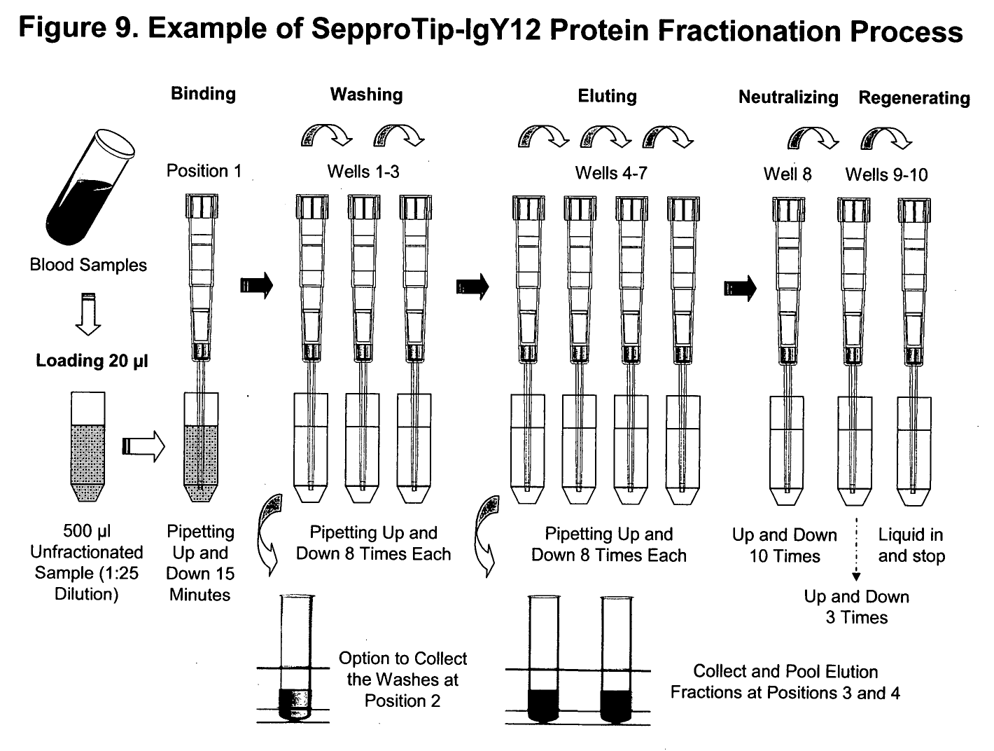

IgY-Microbead Tip (SepproTip) Design is based upon the microtip type of PSS Bio Instruments. FIGS. 7 and 8 shows SepproTip-IgY12 design, specification, and photographs of SepproTip-IgY12 prototype products mounted to PSS Bio Instruments' automated system. The example tip size is 500 μl packed bed volume. As diagrammed in FIG. 9, the process flow was developed and successfully tested for SepproTip-IgY12 application in fractionating human plasma (below).

2. Test of SepproTip-HSA.

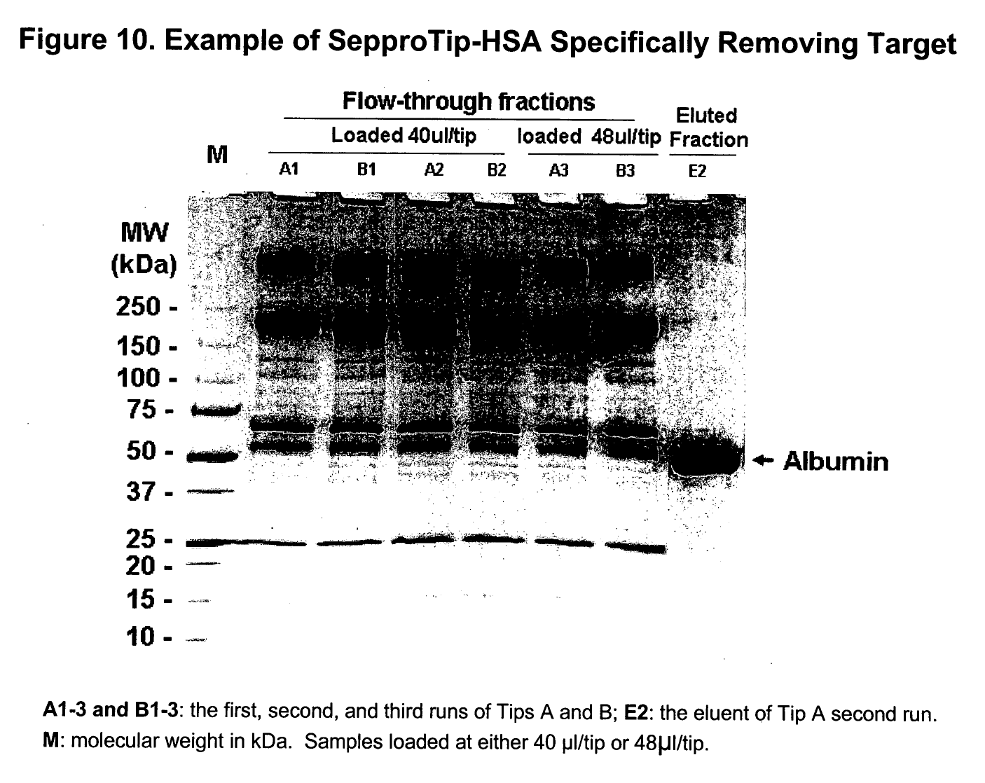

To analyze the capacity and fractionation efficiency of SepproTip-IgY, SepproTip-HSA was used to remove HSA in human plasma. Two volumes (40 μl and 48 μl) of samples were loaded to SepproTip-HSA. The fractions of SepproTip separation were analyzed by SDS-PAGE (FIG. 10). ELISA assay was used for analyzing the flow-through fractions to determine the capture and removal efficiency. A2: 40 μl sample loaded, HSA depleted at 99.7%; A3: 48 μl sample loaded, HSA depleted at 99.6%; B2: 40 μl sample loaded, HSA depleted at 99.7%; B3: 48 μl sample loaded, HSA depleted at 99.3%.

3. Test of SepproTip-IgY12.

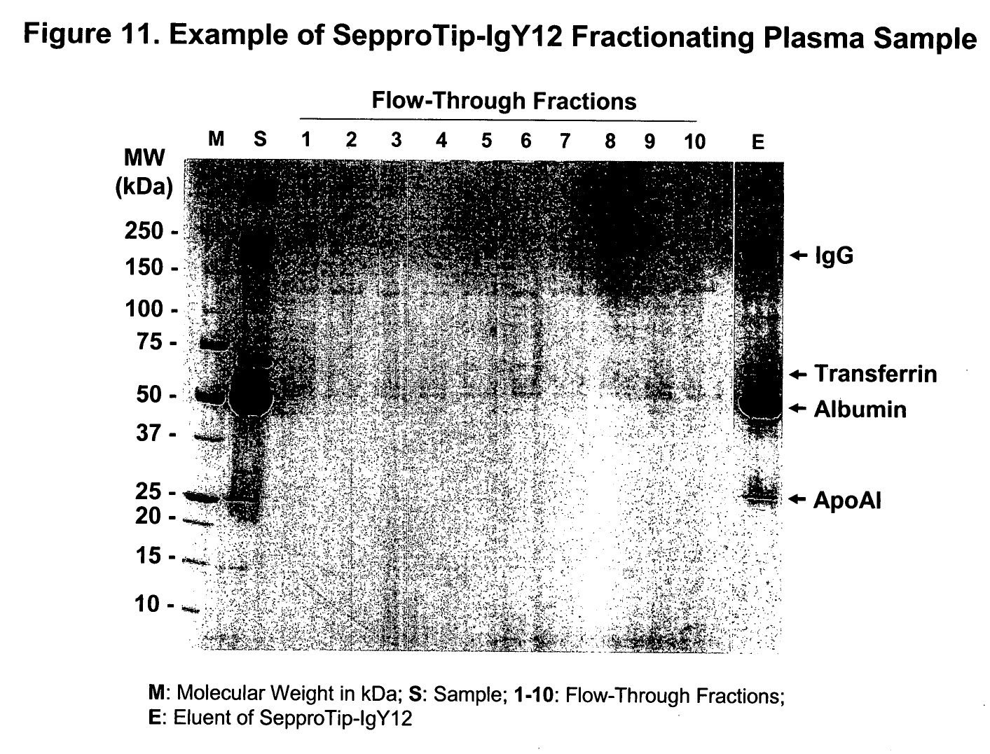

To further analyze the specificity, capacity, efficiency and reproducibility of immunoaffinity fractionation, SepproTip-IgY12 (500 μl) was used to process 20 μl human plasma. Fractions of SepproTip-IgY12 were resolved and visualized by SDS-PAGE. Shown in FIG. 11 are the flow-through fractions from 10 consecutive runs and one representative elution fraction. Based on observation and experience of previous results for human plasma depletion, it is clear that SepproTip-IgY12 efficiently depleted abundant proteins from human plasma in flow-through fractions, and can be regenerated at least 10 times for multiple usages without any reduction in specificity, capacity, reproducibility, and depletion efficiency.

### Example 3

### Preparation of IgY Microbead Mixture with Different Approaches

Mixture of immobilized IgY can be at least three types: (a) individual IgY covalently linked to a solid support first, then mixed artificially in a certain ratio; (b) individual IgY artificially mixed in a certain ratio first, then covalently link to a solid support; and (c) mixture of IgY generated naturally in an immune host system based upon a complex antigen.

1. Preparation of Premixed IgY Microbeads (IgYm12).

To prepare immobilized IgY by using the approach of (b), individual IgY antibodies can be mixed in the ratio needed, then linked to a solid support. The groups of IgY antibodies can be mixed in a ratio for optimized immunoaffinity separation or enrichment of target proteins. One example is to conjugate the premixed 12IgY antibodies through this process. The 12IgY antibodies against HSA, IgGFc, Fibrinogen, Transferrin, IgA, α2-Macroglobulin, IgM, α1-Antitrypsin, Haptoglobin, Apolipoprotein A-I, Apolipoprotein A-II, and α1-Acid Glycoprotein are first mixed in a ratio based on the relative abundance of these 12 proteins in serum/plasma and the capacity of individual IgY microbeads. The mixed population of antibodies is then oxidized with sodium meta-periodate (5 mg/ml) at room temperature for 30 minutes, followed by dialysis against 0.1 M Phosphate Buffered (pH 7.1) to remove residual oxidant. Oxidized IgY antibodies are incubated with UltraLink® Hydrazide beads (Pierce Product No. 53149) to obtain conjugation ratios of 10 to 15 mg IgY/ml beads. Conjugation is carried out at 4° C. overnight with rotation. After conjugation, the IgY-coupled microbeads are thoroughly washed with 1M NaCl, followed by Tris-Buffered Saline (TBS, 10 mM Tris-HCl, 0.15 M NaCl, pH 7.4), and stored as a 50% slurry in TBS with 0.02% NaN₃ at 4° C. The premixed IgY12 microbeads are called IgYm12.

2. Comparison of Separation Efficiency of Two Types of SepproTip-IgY.

To test efficiency of IgY immunoaffinity separation based upon different types of immobilized IgY, IgY antibodies were covalently linked to a solid support via two types of preparation. One type is SepproTip-IgY12, prepared by linking 12 individual IgY antibodies covalently to beads first, then mixing them in a certain ratio. Another type is SepproTip-IgYm12, prepared by mixing 12 individual IgY antibodies in a certain ratio first, then covalently linking them to beads. Both tips (500 μl bed size) were tested under the same condition to fractionate 20 μl of human plasma. The results demonstrate that SepproTip-IgYm12 has better capture efficiency and capacity (FIG. 11). It is not clear why type IgYm12 microbeads showed better performance than type IgY 12 microbeads when the same amount of individual IgY antibodies were used in these two types. We believe it is homogeneity of individual beads that gives superior performance in terms of antigen capturing and releasing from the beads.

### Example 4

### Sequential Immunoaffinity Partitioning (SIP) Using Reiterated SuperMix or SepproTip Method

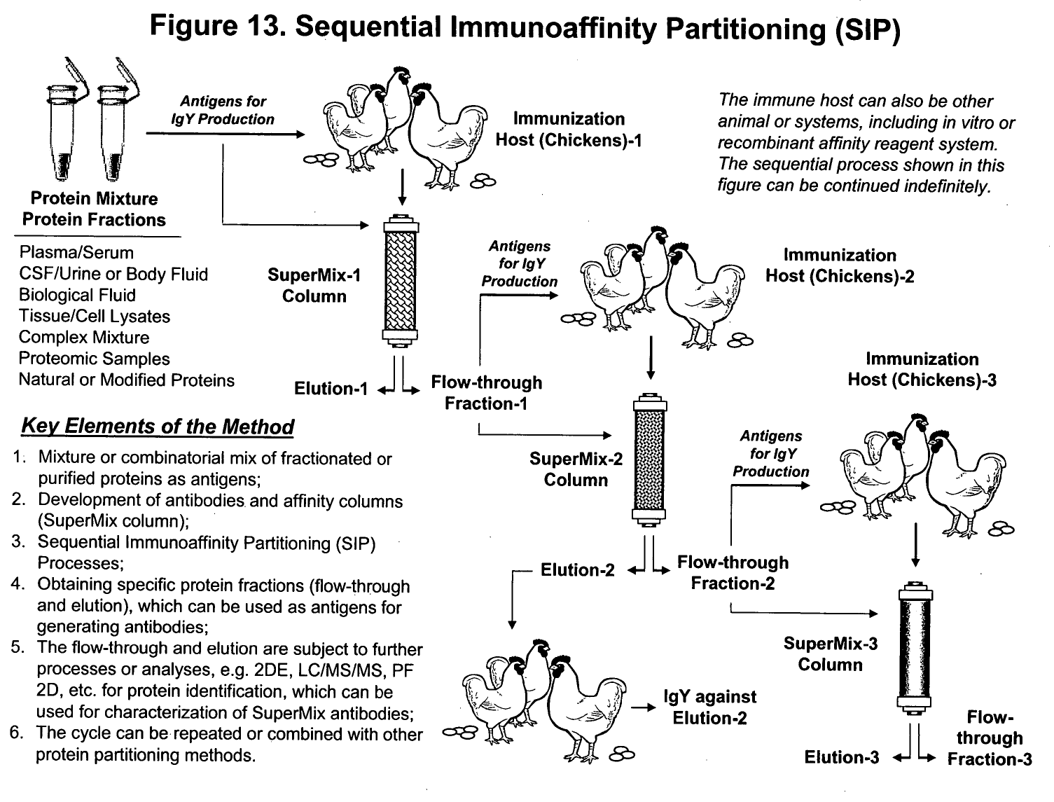

Shown in FIG. 13 is a process of successive fractionation of protein mixtures to access lower and lower abundance proteins. A complex mixture is first used as antigens (or immunogens) to immunize chickens (Immunization Host-1) to develop a SuperMix-1 column (as shown in FIG. 18). The initial complex mixture is applied to the SuperMix-1 column and results in Elution-1 and Flow-Through-1. The latter Flow-Through-1 fraction is then used as antigens to immunize additional chickens (Immunization Host-2) to generate a SuperMix-2 column. The Flow-Through-1 is then applied to the SuperMix-2 column and results in Elution-2 and Flow-Through-2. The process can be repeated until the original complex mixture is fully partitioned. The eluted fractions can also be used as antigens for immunization to generate a plurality of antibodies, wherein these antibodies specifically bind to the eluted fractions and can be used as SuperMix for partitioning antigens in the eluted fractions from a proteome or sub-proteome or for enriching certain antigens or biological materials therefrom. The SuperMix column format described above (and in FIG. 13) in an SIP process can be readily replaced with SepproTip format. The latter can be put in a multiplex and high-throughput setting to enhance robustness, reproducibility, reliability, precision and accuracy. The types of samples that can be used for the SIP processes and host system that can be used for developing affinity reagents are listed. The SIP processes can also be combined with other fractionation and analytical methods such as 2DE, LC/MS/MS, PF 2D, etc.

All patents, patent applications, provisional applications, and publications referred to or cited herein are incorporated by reference in their entirety to the extent that they are not inconsistent with the explicit details of this specification.

We claim:

1. An immunoaffinity separation composition for separating or enriching one or more targets in a complex mixture comprising one or more affinity reagents linked to a solid support and oriented in a manner to facilitate the activity of the affinity reagents wherein said affinity reagents are capable of binding specific targets by affinity recognition and said solid support is capable of mediating separation of the affinity reagent-target complex from the mixture containing non-specific targets.

2. The immunoaffinity separation composition of claim 1 wherein the affinity reagents are IgY polyclonal antibodies having an Fc region and Fab regions, proteins, recombinant proteins, peptides, nucleotides, polymers or a mixture thereof and the target is an antigen.

3. The immunoaffinity separation composition of claim 2 wherein the affinity reagents are IgY antibodies, having an Fc region and Fab regions, that are covalently linked to the solid support with a bond to the Fc region of the polyclonal IgY antibodies wherein said polyclonal IgY antibodies are made by immunizing and boosting a bird with antigen(s) and said antibodies specifically bind with said antigen(s) through the Fab regions.

4. The immunoaffinity separation composition of claim 3 wherein the bird is a chicken.

5. The immunoaffinity separation composition of claim 3 further comprising one or more antigens present in the complex mixture wherein said antigens are affinity recognized and bound by the Fab regions of the IgY antibodies.

6. The immunoaffinity separation composition of claim 3 wherein the antigen is a protein, a peptide, a protein-protein complex, a protein-nucleotide complex, a protein-sugar/lipid complex, a biological complex, a nucleotide, a cell or a subcellular organelle, or a microorganism, all of which can induce antibodies in the bird.

7. The immunoaffinity separation composition of claim 3 wherein the support is covalently linked with one or more populations of polyclonal IgY antibodies wherein each population of polyclonal antibodies binds with a different human protein.

8. The affinity separation composition of claim 7 that contains 2 or more populations of antibodies covalently linked to the solid support wherein said populations of antibodies are (a) independently linked to said solid support respectively and then mixed in a given ratio to form the immunoaffinity separation composition or (b) mixed in the given ratio and then linked to the solid support together.

9. A method of affinity separating at least one target protein in a complex protein mixture which comprises the steps of:
a. providing a complex protein mixture which contains at least one target protein,
b. contacting the complex protein mixture with an immunoaffinity separation composition according to claims 1-8 wherein at least one target protein in the complex mixture specifically binds the immunoaffinity separation composition, and
c. recovering an elution fraction, wherein the elution fraction comprises at least one target protein which is bounded to the immunoaffinity separation composition.

10. The method of claim 9 further comprising a step of recovering a flow-through protein fraction, wherein the flow-through fraction is a remaining complex protein mixture after the complex protein mixture has been contacted with but is separated from the immunoaffinity separation composition and the concentration of at least one target protein which has been bounded to the immunoaffinity separation composition in the flow-through fraction is less than that in the complex protein mixture.

11. The method of claim 9 wherein the complex protein mixture is plasma, serum, derived from tissue, cerebrospinal fluid, bronchial alveolar lavage, vitreous humor, nipple aspirate, urine, a flow-through fraction or an elution fraction thereof.

12. The method of claim 9 wherein the target protein is one or more proteins selected from the group consisting of Highly-Abundant Protein (HAP), Moderately-Abundant Protein (MAP), and Low-Abundant Protein (LAP), wherein HAP is selected from the group consisting of Albumin, IgG, Fibrinogen, Transferrin, IgA, α2-Macroglobulin, IgM, α1-Antitrypsin, Haptoglobin, α1-Acid Glycoprotein, Apolipoprotein A-I, Apolipoprotein A-II and High Density Lipoprotein.

13. The method of claim 9 wherein the protein mixture is a human protein mixture.

14. The method of claim 9 wherein the elution fraction is subject to identification, analysis, or characterization.

15. The method of claim 10 wherein a protein in the flow through protein mixture is subject to identification, analysis, or characterization.

16. The method of claim 14 or 15 wherein identification, analysis, or characterization includes 2DE, PF 2D, and LC/MS/MS.

17. A method of fractionating a mixture of biological materials comprising the steps of:
a. immunizing a first host using a first antigen or a first mixture of biological materials;
b. obtaining a plurality of first IgY polyclonal antibodies from the first host, wherein the first IgY polyclonal antibodies, affinity-purified by the first antigen or the first mixture of biological materials, bind substantially to the first antigen or the first mixture of materials;
c. covalently conjugating the first IgY antibodies on a support to form a first affinity separation composition;
d. contacting the first antigen or the first mixture of proteins with the first affinity separation composition; and
e. collecting a first flow-through fraction.

18. The method of claim 17 further comprising the steps of:
a. immunizing a second host using a second mixture of biological materials which comprises the first flow-through fraction;
b. obtaining a plurality of second IgY polyclonal antibodies from the second host, wherein the second IgY polyclonal, affinity-purified by the second mixture of biological materials, bind substantially to the second mixture of biological materials;
c. covalently conjugating the second IgY antibodies to a support to form a second affinity separation composition;
d. contacting the first flow-through fraction with the second affinity separation composition; and
e. collecting a second flow through fraction.

19. The method of claim 18 further comprising the step of repeating the steps of (a) through (e) in claim 18.

20. The method of claim 17 further comprising the step of collecting a first elution fraction, wherein the first elution fraction contains elements or proteins in the first antigen or mixture of biological materials that bind to the first affinity separation composition.

21. The method of claim 18 further comprising the step of colleting a second elution fraction, wherein the second elution fraction contains elements or proteins in the second or mixture of biological materials that bind to the second affinity separation composition.

22. The method of claims 17-21 wherein the biological materials comprise proteins.

23. The method of claim 22 wherein the proteins include HAPs, MAPs, and LAPs.

24. The method of claim 23 wherein HAPs are fractionated from MAPs and/or LAPs.

25. The method of claim 24 wherein MAPs are fractionated from HAPs and/or LAPs.

26. The method of claim 25 wherein LAPs are fractionated from HAPs and/or MAPs.

27. The method of claim 17 wherein the first host is an animal.

28. The method of claim 27 wherein the animal is a bird.

29. The method of claim 17 wherein the first flow through fraction is subject to identification, analysis, or characterization.

30. The method of claim 18 wherein the second flow through fraction is subject to identification, analysis, or characterization.

31. The method of claim 20 wherein the first elution fraction is subject to identification, analysis, or characterization.

32. The method of claim 21 wherein the second elution fraction is subject to identification, analysis, or characterization.

33. The methods of claim 29-32 wherein identification, analysis, or characterization includes 2DE, PF 2D, and LC/MS/MS.

34. The method of claim 17 wherein the antibodies are oriented in conjugation with the support such that the Fc portions of the antibodies are covalently conjugated to the support.

35. A high-throughput method of fractioning a first mixture of biological materials and a second mixture of biological materials or enriching a first target protein of the first mixture and a second target protein of the second mixture, comprising:
a. contacting a first mixture of biological materials with a first immunoaffinity separation composition and a second mixture of biological materials with a second immunoaffinity separation composition; wherein the first target protein binds to the first immunoaffinity separation composition and the second target protein binds to the second immunoaffinity separation composition;
b. collecting a first flow through fraction and a second flow through fraction; wherein the concentration of the first target protein in the first flow through fraction is less than in the first mixture and the concentration of the second target protein in the second flow through fraction is less than in the second mixture.

36. The high-throughput method of claim 35 further comprising a step of collecting a first elution fraction and a second elution fraction, wherein the first elution fraction comprises a first target protein and the second elution fraction comprises a second targeted protein.

37. The high-throughput method of claim 35 wherein the method is conducted through automation.

38. The high-throughput method of claim 35 wherein the first and second mixtures are in different regions of a same substrate.

39. The high-throughput method of claim 35 wherein the first and second immunoaffinity separation compositions are in different regions of a same substrate.

40. The high-throughput method of claim 35 wherein the first and second immunoaffinity separation compositions are in different holders which are arranged in array, multiplex, or parallel.

41. The high-throughput method of claim 40 wherein the holders are columns, pipette tips, microtips, tubes, or fluidic channels.

42. The high-throughput method of claim 35 wherein the first and second flow through fractions are subject to identification, analysis, or characterization.

43. The high-throughput method of claim 36 wherein the first and second elution fractions are subject to identification, analysis, or characterization.

44. The high-throughput method of claim 42 or 43 wherein identification, analysis, or characterization includes 2DE, PF 2D, and LC/MS/MS.

45. The high-throughput method of claim 35 wherein the first mixture and the second mixture are fractioned or enriched simultaneously.

46. The high-throughput method of claim 35 wherein the first flow through fractions and the second flow through fraction are collected simultaneously.

47. The high-throughput method of claim 36 wherein the first elution fraction and the second elution fraction are collected simultaneously.

48. An array of immunoaffinity separation compositions for high-throughput method comprising a first immunoaffinity separation composition and a second immunoaffinity separation composition.

49. The array of immunoaffinity separation compositions of claim 48 wherein the first and second immunoaffinity separation compositions are in different regions of a same substrate.

50. The array of immunoaffinity separation compositions of claim 48 wherein the first and second immunoaffinity separation compositions are in different holders which are arranged in array, multiplex, or parallel.

51. The array of immunoaffinity separation compositions of claim 50 wherein the holders are columns, pipette tips, microtips, tubes, or fluidic channels.

52. A kit comprising an immunoaffinity separation composition or a plurality of immunoaffinity separation compositions.

53. The kit of claim 52 wherein the immunoaffinity separation composition is on a substrate or in a holder.

54. The kit of claim 53 wherein the holder is a column, a pipette tip, a microtip, a tube, or a fluidic channel.

55. The kit of claim 52 wherein a plurality of immunoaffinity separation compositions are in different regions of a substrate or in different holders which are arranged in array, multiplex, or parallel.

56. The kit of claim 55 wherein the holders are columns, pipette tips, microtips, tubes, or fluidic channels.
